(19) United States
(12) Patent Application Publication
(10) Pub. No.: US 2014/0121767 A1
(43) Pub. Date: May 1, 2014

(54) METHOD AND SYSTEM FOR PROVIDING A TORIC INTRAOCULAR LENS

(71) Applicant: NOVARTIS AG, Basel (CH)

(72) Inventor: MICHAEL J. SIMPSON, ARLINGTON, TX (US)

(73) Assignee: NOVARTIS AG, Basel (CH)

(21) Appl. No.: 14/034,639

(22) Filed: Sep. 24, 2013

Related U.S. Application Data
(60) Provisional application No. 61/720,669, filed on Oct. 31, 2012.

(51) Int. Cl. A61F 2/16

(52) U.S. Cl. CPC A61F 2/1637
USPC 623/6.23

(57) ABSTRACT

A method and system provide an ophthalmic device and treat a patient using the ophthalmic device. The ophthalmic device includes an ophthalmic lens having an anterior surface, a posterior surface and an optic axis. At least one of the anterior surface and the posterior surface is an aspheric surface. The aspheric surface has a toricity configured to spread retroreflected light incident in a plurality of directions canted from the optic axis. In one aspect, the method includes selecting the ophthalmic device for implantation in an eye of the patient and implanting the ophthalmic device in the patient's eye.

This application claims the priority of U.S. Provisional Patent Application No. 61/720,669 filed on Oct. 31, 2012, the contents of which are incorporated herein by reference in their entirety.

### BACKGROUND

Intraocular lenses (IOLs) are implanted in patients' eyes either to replace a patient's lens or, in the case of a phakic IOL, to complement the patient's lens. For example, the IOL may be implanted in place of the patient's lens during cataract surgery. Alternatively, a phakic IOL may be implanted in a patient's eye to augment the optical power of the patient's own lens. FIG. 1 depicts a conventional system 10 in which a conventional IOL 20 has been placed in the eye 12 of a patient, replacing the patient's lens. The conventional IOL 20 is a spherical lens having an optic axis 22, anterior surface 24, and posterior surface 26. The conventional IOL refracts light 14 incident to the patient's eye 20, in order to form an image on the retina improving the patient's vision.

Although the conventional IOL 20 functions, the IOL may also retroreflect light 14 incident to the patient's eye 12. In some instances, the retroreflected light is aligned along a single direction. This is shown as occurring from the anterior surface 24 of the conventional IOL 20 in FIG. 1. Typically this phenomenon occurs when the radius of the wavefront converging from the cornea (not explicitly depicted) matches radius of curvature of the anterior surface 24 of the conventional IOL 20. However, a similar phenomenon may also occur from the posterior surface 26 of the conventional IOL 20. Although only a small percentage of the energy incident to the eye 12 may be retroreflected, this light may be visible to observers. For example, the retroreflected light 14 may be visible for small, bright light sources. The retroreflected light 14 appears to originate in the patient's eye 12. Although harmless to the patient, the retroreflected light may still be disturbing to observers. Thus, this phenomenon is known as “scary eye”.

A conventional method for reducing the occurrence of retroreflected light is to adjust the radius of curvature of the anterior surface 24 or posterior surface 26. However, this change may not be possible for all lens powers. Thus, for certain conventional IOLs 24, retroreflection of light may still be an issue for patients.

Accordingly, what is needed is a system and method for reducing the occurrence of “scary eye” in patients.

### BRIEF SUMMARY OF THE INVENTION

A method and system provide an ophthalmic device and treat a patient using the ophthalmic device. The ophthalmic device includes an ophthalmic lens having an anterior surface, a posterior surface and an optic axis. At least one of the anterior surface and the posterior surface is an aspheric surface. The aspheric surface has a toricity configured to spread retroreflected light incident in a plurality of directions canted from the optic axis. In one aspect, the method includes selecting the ophthalmic device for implantation in an eye of the patient and implanting the ophthalmic device in the patient's eye.

### BRIEF DESCRIPTION OF SEVERAL VIEWS OF THE DRAWINGS

FIG. 1 depicts a conventional ophthalmic device as used in a patient's eye.

FIG. 2 depicts a plan view of an exemplary embodiment of an ophthalmic device.

FIGS. 3-4 depict side and top views of an exemplary embodiment of a portion of an ophthalmic device as used in a patient's eye.

FIG. 5 depicts a perspective view of an exemplary embodiment of a portion of an ophthalmic device.

FIG. 6 depicts the spread in intensity of reflected light from a spherical lens and a toric lens.

FIGS. 7-8 depict side and top views of another exemplary embodiment of a portion of an ophthalmic device as used in a patient's eye.

FIGS. 9-10 depict side and top views of another exemplary embodiment of a portion of an ophthalmic device as used in a patient's eye.

FIGS. 11-12 depict side and top views of another exemplary embodiment of a portion of an ophthalmic device as used in a patient's eye.

FIG. 13 is flow chart depicting an exemplary embodiment of a method for utilizing an ophthalmic device.

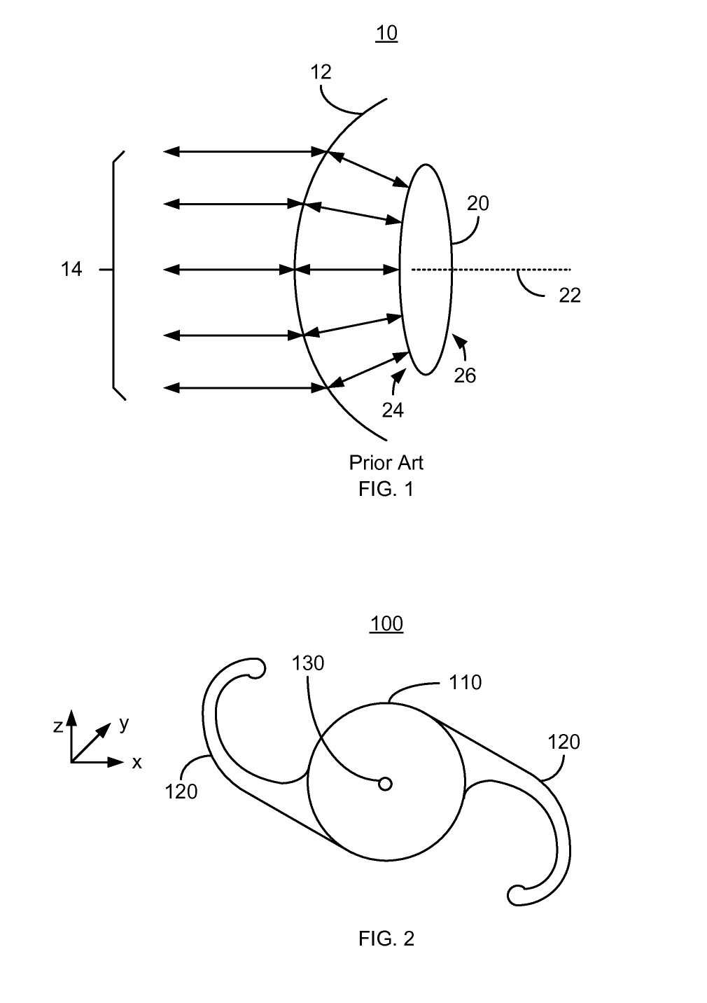

### DETAILED DESCRIPTION OF THE PREFERRED EMBODIMENTS

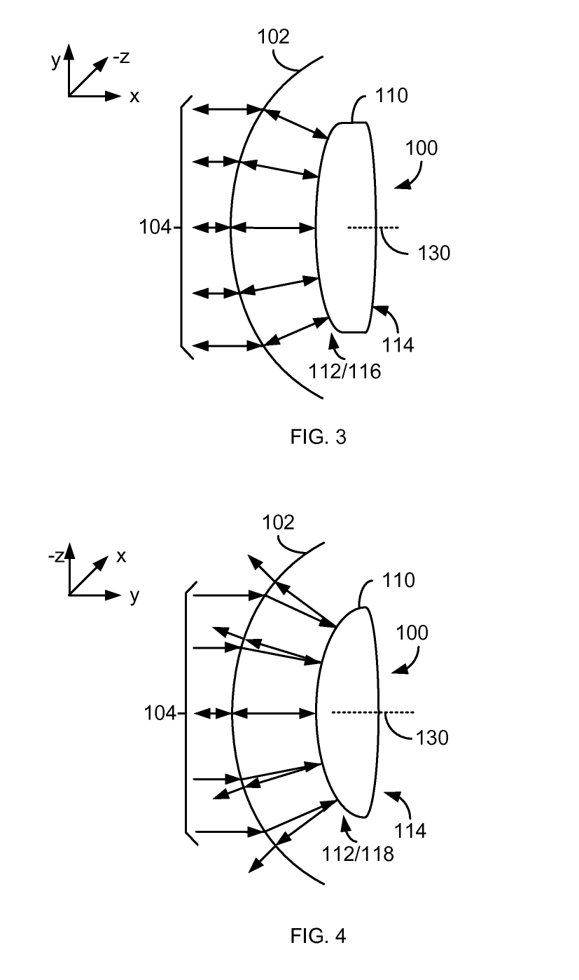

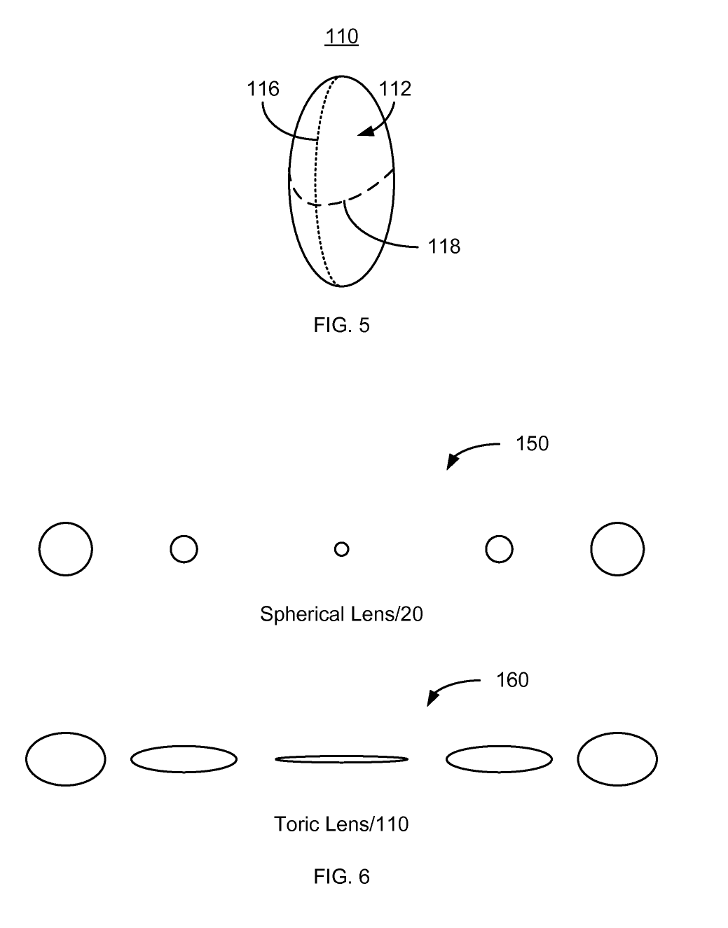

FIGS. 2-5 depict various views of an exemplary embodiment of an ophthalmic device 100 that may be used as an IOL. FIG. 2 depicts a plan view of the ophthalmic device 100, which includes an ophthalmic lens 110, haptics 120, and an optic axis 130. FIGS. 3-4 depict side and top views of the ophthalmic lens 110 of the ophthalmic device 100 as used in a patient's eye 102. FIG. 5 depicts a perspective view of the ophthalmic lens 110. For clarity, FIGS. 2-5 are not to scale.

Haptics 120 are used to hold the ophthalmic device 100 in place in a patient's eye 102. However, in other embodiments, other mechanism(s) might be used to retain the ophthalmic device in position in the eye 102. For clarity, the haptics are not depicted in FIGS. 3-5. Although the ophthalmic lens 102 is depicted as having a circular cross section in the plan view of FIG. 2, in other embodiments, other shapes may be used.

As can be seen in FIGS. 3-5, the ophthalmic lens 110 has an anterior surface 112 configured to be closer to the front of the patient's eye (e.g. the cornea) and a posterior surface 114. In the embodiment shown, the anterior surface 112 is aspheric, while the posterior surface 114 is spheric. More specifically, the anterior surface 112 is toric. Thus, as can be seen in FIG. 5, the anterior surface 112 has meridians 116 and 118 that are orthogonal. Because the anterior surface 112 is toric, the meridians 116 and 118 have different radii of curvature. The toricity of the anterior surface 112 may be characterized by the difference in the radii of curvature. For example, in some embodiments, the anterior surface 112 has at least 1.5 and not more than six diopters of astigmatism. In some such embodiments, the anterior surface 112 has at least two diopters of astigmatism. However, in other embodiments, the toricity of the anterior surface 112 may be different. Further, although shown as spheric, the posterior surface 114 may have another shape. For example, the posterior surface 114 may be toric. In some embodiments, the combination of the shapes of the anterior surface 112, the shape of the posterior surface 114 and/or other characteristics of the ophthalmic lens 110 (such as thickness) may correct for various vision issues of the patient. For example, the ophthalmic lens 110 may correct for near-sightedness, far-sightedness and/or astigmatism.

FIGS. 3 and 4 depict the ophthalmic lens 110 along different meridians 116 and 118, respectively. In FIGS. 3 and 4, the ophthalmic lens 110 has been implanted in an eye 102 of a patient and may be retained in place using haptics 120 (not shown in FIGS. 3-5). The anterior surface 112 of the ophthalmic lens 110 retroreflects light. Although not depicted as doing so, the posterior surface 114 may also retroreflect light. The view shown in FIG. 3 is along the meridian 116 of the anterior surface 112. The view shown in FIG. 4 is along the meridian 118 of the anterior surface 112. Thus, the radius of curvature for the section of the ophthalmic lens 110 shown in FIG. 3 is greater than for the section of the ophthalmic lens 110 shown in FIG. 4.

The radius of curvature for the meridian 116 happens to match the radius of curvature for the wavefront of the light 104 incident on the anterior surface 112. Thus, as is shown in FIG. 3, the light 104 is retroreflected back substantially along a single direction that happens to be substantially parallel to the optic axis 130. If the anterior surface 112 were spherical and had the radius of curvature of the meridian 116, the patient would be subject to “scary eye”. However, the other meridian 118 has a different radius of curvature that does not match the radius of curvature for the wavefront of the light 104. Along the meridian 118, therefore, light reflected by the anterior surface 112 is spread in directions away from the optic axis 130 and away from the direction at which light 104 was incident to the ophthalmic lens 110. This situation is shown in FIG. 4.

FIG. 6 depicts spot diagrams 150 and 160 for light retroreflected from a spherical lens (not shown) and a toric lens such as the ophthalmic lens 110. The spot diagrams 150 and 160 are for explanatory purposes only and not meant to reflect a particular real-world ophthalmic device. Referring to FIGS. 2-6, each spot in the spot diagrams 150 and 160 includes the same power as the spot directly above/below. The spot diagram 150 corresponds to a spherical lens (such as the conventional lens 20) having a radius of curvature corresponding to the meridian 116. The spot diagram 160 corresponds to the ophthalmic lens 110 in which the meridians 116 and 118 have different radii of curvature. The meridian 116 may be considered to retroreflect light vertically, while the meridian 118 may be considered to retroreflect light horizontally in the spot diagram 160. As can be seen in comparing the spot sizes, the toric lens 110 spreads the same power over a larger area because reflections along the meridian 118 tend to be spread over multiple directions. In some cases, the area of a spot for the toric lens diagram 160 may be at least ten times that of the corresponding spot in the spherical lens diagram 150. Thus, the ophthalmic lens 110 may have a lower intensity (power divided by solid angle) of retroreflected light than a conventional spherical lens.

The ophthalmic device 100 including ophthalmic lens 110 may reduce the effect of “scary eye” for a patient. Because the ophthalmic lens 110 has a toricity (different radii of curvature for the meridians 116 and 118), light incident on the anterior surface 112 generally does not have a wavefront that matches the radius of curvature for both meridians 116 and 118. This may be accomplished by designing the meridians 116 and 118 to have radii of curvature that differ by at least 1.5 diopters, at least 2.0 diopters, or more. Because of the toricity of the anterior surface 112, the wavefront does not match the radii of curvature for both meridians 116 and 118 and the anterior surface 112 spreads retroreflected light in multiple directions. Thus, a lower intensity of retroreflected light may be observed by individuals viewing the patient and the effect of “scary eye” reduced. Although the toricity and attendant correction of “scary eye” is described in the context of the anterior surface 112, the posterior surface 114, or both the anterior surface 112 and the posterior surface 114 may be similarly configured.

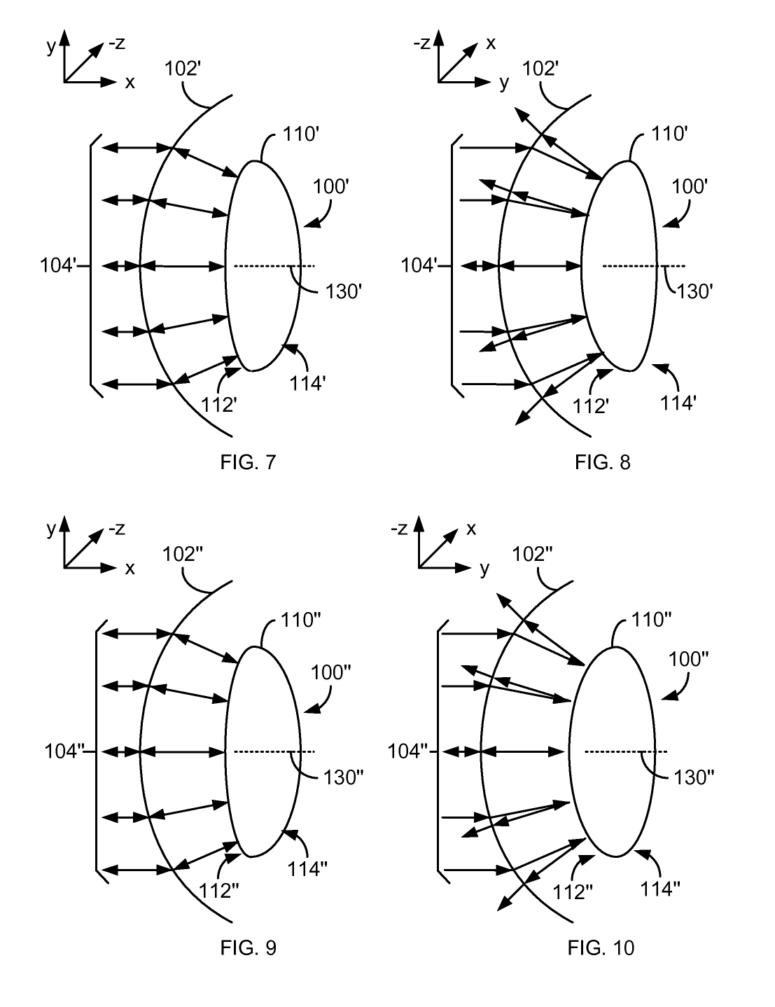

FIGS. 7 and 8 depict side and top views, respectively, of another exemplary embodiment of an ophthalmic device 100′. For clarity, FIGS. 7-8 are not to scale. The ophthalmic device 100′ corresponds to the ophthalmic device 100. Similar components have analogous labels. The ophthalmic device 100′ includes an ophthalmic lens 110′ having an anterior surface 112′, posterior surface 114′, and optic axis 130′ that corresponds to ophthalmic lens 110 having anterior surface 112, posterior surface 114 and optic axis 130, respectively. Thus, the components 110′, 112′, 114′ and 130′ have a similar structure and function to the components 110, 112, 114 and 130, respectively. The ophthalmic device 100′ may also have haptics (not shown in FIGS. 7-8) or other mechanism for retaining the ophthalmic device in place that correspond to the haptics 120. In FIGS. 7 and 8, the ophthalmic lens 110′ has been implanted in an eye 102′ of a patient and may be retained in place using haptics or another mechanism. The ophthalmic device 100′ utilizes the curvature(s) of the posterior surface 114′ to account for the toricity of the anterior surface introduced to reduce “scary eye” and/or to address other vision issues.

In the embodiment shown, the anterior surface 112′ and the posterior surface 114′ are both aspheric. The anterior surface 112′ is toric. Thus, the anterior surface 112′ has meridians (not shown) that are orthogonal. Because the anterior surface 112′ is toric, the meridians have different radii of curvature. In some embodiments, the anterior surface 112′ has at least 1.5 and not more than six diopters of astigmatism. In some such embodiments, the anterior surface 112′ has at least two diopters of astigmatism. However, in other embodiments, the toricity of the anterior surface 112′ may be different. The posterior surface 114′ is also toric. Thus, like the anterior surface 112, the posterior surface 112′ is characterized by different radii of curvature along different meridians, which may be perpendicular. However, the toricity of the posterior surface 114′ is different from the toricity of the anterior surface 112′.

Because side and top views are shown, FIGS. 7 and 8 depict the ophthalmic lens 110′ along different meridians. The anterior surface 112′ of the ophthalmic lens 110′ retroreflects light. Although not depicted as doing so, the posterior surface 114′ may also retroreflect light. For the meridian depicted in FIG. 7, the radius of curvature for the meridian happens to match the radius of curvature for the wavefront of the light 104′ incident on the anterior surface 112′. Thus, as is shown in FIG. 7, the light 104′ is retroreflected back substantially along a single direction that happens to be substantially parallel to the optic axis 130′. If the anterior surface 112′ were spherical, the patient would be subject to “scary eye”. However, for the meridian depicted in FIG. 8, the anterior surface 112′ does not retroreflect the light along a single direction. Instead, light reflected by the anterior surface 112′ is spread in directions away from the optic axis 130′ and away from the direction at which light 104′ was incident to the ophthalmic lens 110′ in FIG. 8. Thus, “scary eye” may be reduced or eliminated through the configuration of the anterior surface 112′.

In addition, the posterior surface 114′ is configured to account for the toricity of the anterior surface 112′ introduced to reduce “scary eye”. Thus, the lens 110′ as a whole functions as desired. For example, in some embodiments, the lens 110′ is desired to function as a spherical lens. In such an embodiment, the meridians for the posterior surface 114′ are opposite to those of the anterior surface 112′. In such embodiments, the radius of curvature along one meridian for the anterior surface 112′ is the same as the radius of curvature along an orthogonal meridian for the posterior surface 114′. Thus, the combination of the shape of the anterior surface 112′ and the shape of the posterior surface 114 can reduce the incidence of “scary eye”, yet functions as a spherical lens. Further, the posterior surface 114′ may provide correction for astigmatism, near-sightedness, far-sightedness and/or other issues with the patient's vision. For example, the toricity of the posterior surface 114′ may be such that the entire lens 110′ (e.g. anterior surface 112′ and posterior surface 114′ together) together correct for astigmatism of the patient. This toricity would be in addition to the toricity of the anterior surface 112′ that reduces “scary eye”. Thus, the ophthalmic device 100′ including ophthalmic lens 110′ may reduce the effect of “scary eye” for a patient while addressing other aspects of the patient's vision. Although the toricity and attendant correction of “scary eye” is described in the context of the anterior surface 112′, the posterior surface 114′ or both the surfaces 112′ and 114′ may be similarly configured.

FIGS. 9 and 10 depict side and top views, respectively, of another exemplary embodiment of an ophthalmic device 100″. For clarity, FIGS. 9-10 are not to scale. The ophthalmic device 100″ corresponds to the ophthalmic devices 100 and/or 100′. Similar components have analogous labels. The ophthalmic device 100″ includes an ophthalmic lens 110″ having an anterior surface 112″, posterior surface 114″, and optic axis 130″ that corresponds to lens 100/100′ having anterior surface 112/112′, posterior surface 114/114′ and optic axis 130/130′, respectively. Thus, the components 110″, 112″, 114″ and 130″ have a similar structure and function to the components 110/110′, 112/112′, 114/114′ and 130/130′, respectively. The ophthalmic device 100″ may also have haptics (not shown) or other mechanism for retaining the ophthalmic device in place that correspond to the haptics 120. In FIGS. 9 and 10, the ophthalmic lens 110″ has been implanted in an eye 102″ of a patient and may be retained in place using haptics or other mechanism. The ophthalmic device 100″ utilizes the curvature(s) of the posterior surface 114″ and, optionally, the anterior surface 112″ in order to account for the toricity of the anterior surface introduced to reduce “scary eye” and/or to correct other aspect(s) of the patient's vision.

In the embodiment shown, the anterior surface 112″ is aspheric. In some embodiments, the posterior surface 114″ is also aspheric. The anterior surface 112″ is toric. For example, in some embodiments, the anterior surface 112″ has at least 1.5 and not more than six diopters of astigmatism. In some such embodiments, the anterior surface 112″ has at least two diopters of astigmatism. However, in other embodiments, the toricity of the anterior surface 112″ may be different. The posterior surface 114″ may also be toric. Thus, like the anterior surface 112″, the posterior surface 112″ may be characterized by different radii of curvature along different meridians, which may be perpendicular. The toricity of the posterior surface 114″ may be different from the toricity of the anterior surface 112″.

The toricity of the anterior surface 112″ may correct for both “scary eye” and additional issues with the patient's vision. As discussed above for other embodiments and seen in FIGS. 9-10, the radius of curvature for the meridian shown in FIG. 9 happens to match the radius of curvature for the wavefront of the light 104″ incident on the anterior surface 112″. If the anterior surface 112″ were spherical, the patient would be subject to “scary eye”. However, for the meridian depicted in FIG. 10, the anterior surface 112″ does not retroreflect the light along a single direction. Instead, light reflected by the anterior surface 112″ is spread in directions away from the optic axis 130″ and away from the direction at which light 104″ was incident to the ophthalmic lens 110″ in FIG. 10. Thus, “scary eye” may be reduced or eliminated. In addition, the anterior surface 112′ may have an additional toricity that accounts in full or in part for other aspects of the patient's vision. For example, a portion of the total toricity of the anterior surface 112″ may correct for astigmatism in the patient's vision.

The posterior surface 114″ is configured to account for the toricity of the anterior surface 112″ introduced to reduce “scary eye”. Thus, the lens 110″ as a whole functions as desired. For example, the lens 110″ may be desired to both reduce “scary eye” and correct for astigmatism in the patient's vision. In such an embodiment, the meridians for the posterior surface 114″ are opposite to the curvature in the meridians of the anterior surface 112″ that are responsible for the reduction in “scary eye”. However, the portion of the toricity of the anterior surface 112″ that is used to correct for the patient's astigmatism is not opposed by the geometry of the posterior surface 114″. Thus, the ophthalmic device 100″ including ophthalmic lens 110″ may reduce the effect of “scary eye” for a patient while correcting other aspects of the patient's vision.

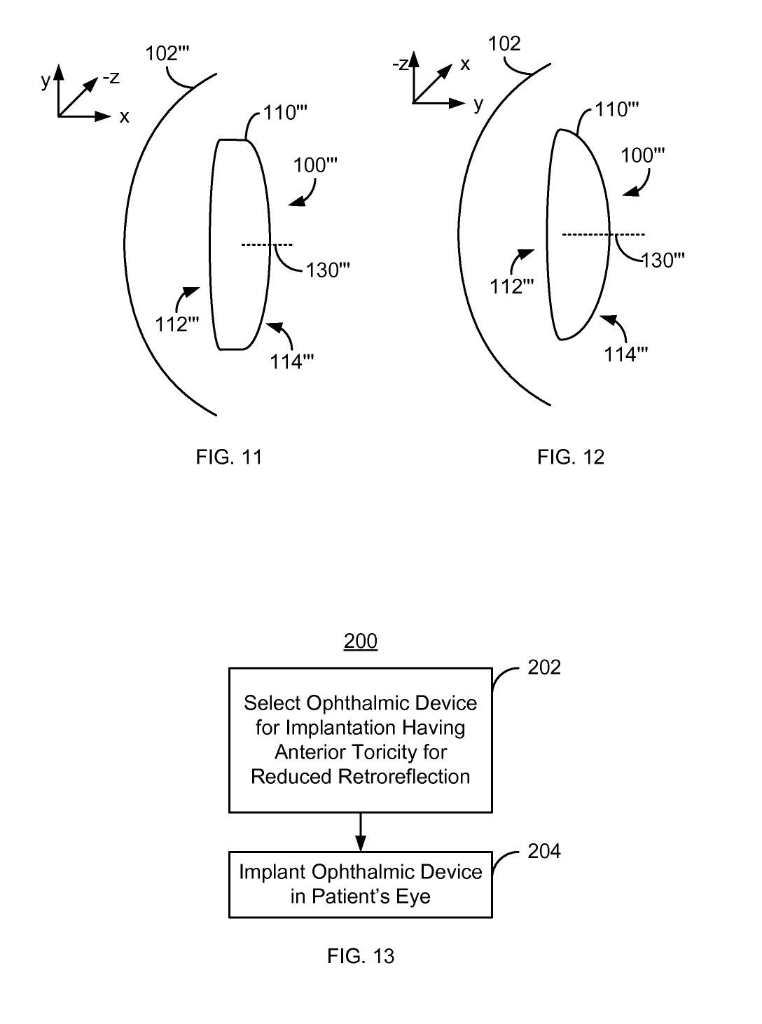

FIGS. 11 and 12 depict side and top views, respectively, of another exemplary embodiment of an ophthalmic device 100″′. For clarity, FIGS. 11-12 are not to scale. The ophthalmic device 100″′ corresponds to the ophthalmic devices 100, 100′ and/or 100″. Similar components have analogous labels. The ophthalmic device 100″′ includes an ophthalmic lens 110″′ having an anterior surface 112″′, posterior surface 114″′, and optic axis 130″′ that corresponds to lens 100/100′/100″ having anterior surface 112/112′/112″, posterior surface 114/114′/114″ and optic axis 130/130′/130″, respectively. Thus, the components 110″′, 112″′, 114″′ and 130″′ have a similar structure and function to the components 110/110′/110″, 112/112′/112″, 114/114′/114″ and 130/130′/130″, respectively. The ophthalmic device 100″ may also have haptics (not shown) or other mechanism for retaining the ophthalmic device in place that correspond to the haptics 120. In FIGS. 11 and 12, the ophthalmic lens 110″′ has been implanted in an eye 102″′ of a patient and may be retained in place using haptics or other mechanism. The ophthalmic device 100″′ utilizes the curvature(s) of the posterior surface 114″ and, optionally, the anterior surface 112″ in order to reduce “scary eye” and/or to correct other aspect(s) of the patient's vision.

In the embodiment shown, the posterior surface 114″′ is aspheric. The anterior surface 112″′ may be spheric or aspheric. The posterior surface 114″ is toric. For example, in some embodiments, the posterior surface 114″′ has at least 1.5 and not more than six diopters of astigmatism. In some such embodiments, the posterior surface 114″ has at least two diopters of astigmatism. However, in other embodiments, the toricity of the posterior surface 114″′ may be different. The anterior surface 112″′ may be toric or, as is shown in FIGS. 11-12, spheric. The toricity of the posterior surface 114″′ may be different from the toricity of the anterior surface 112″′.

The toricity of the posterior surface 114″′ may correct for “scary eye” and, optionally, additional issues with the patient's vision. Thus, the radii of curvature of the posterior surface 114″′ shown in FIGS. 11 and 12 are different. The radius of curvature on the meridian shown in FIG. 11 or 12 may happen to match the radius of curvature for the wavefront of the light (not shown) incident on the posterior surface 114′″. If the posterior surface 114′″ were spherical, the patient would be subject to “scary eye”. However, for the other meridian in FIG. 12 or 11, the radius of curvature of the posterior surface 114″′ does not match the radius of curvature of the wavefront. Instead, light will be spread away from the optic axis upon reflection by the posterior surface 114″′. Thus, “scary eye” may be reduced or eliminated. In addition, the posterior surface 114″′ and/or anterior surface 112″′ may have an additional toricity that accounts in full or in part for other aspects of the patient's vision. For example, a portion of the total toricity of the lens 110″′ may correct for astigmatism in the patient's vision. Further, the anterior surface 112″′ may be configured to account for the toricity of the posterior surface 114″′ introduced to reduce “scary eye”. For example, the anterior surface 112″′ and/or posterior surface 114″′ may be configured in a manner analogous as described above for the posterior surface 114′/114″ and/or anterior surface 112′/112″, respectively. Thus, the lens 110′″ as a whole functions as desired. Thus, the ophthalmic device 100″′ including ophthalmic lens 110″′ may reduce the effect of “scary eye” for a patient while correcting other aspects of the patient's vision.

Thus, ophthalmic devices 100, 100′, 100″, and/or 100″′ may be used to address “scary eye” in patients. In some embodiments, another ophthalmic device including one or more of the characteristics of the ophthalmic devices 100, 100′, 100″, and/or 100″′ may be used to achieve the benefits of the ophthalmic devices 100, 100′, 100″, and/or 100″′. Thus, outcomes for patients may be improved.

FIG. 13 is an exemplary embodiment of a method 200 for treating an ophthalmic condition in a patient. For simplicity, some steps may be omitted, interleaved, and/or combined. The method 200 is also described in the context of using the ophthalmic device 100. However, the method 200 may be used with one or more of ophthalmic devices 100, 100′, 100″, 100″′ and/or an analogous ophthalmic device.

An ophthalmic device 100 for implantation in an eye of the patient is selected, via step 202. The ophthalmic device 100 includes an ophthalmic lens 110 having an anterior surface 112, a posterior surface 114 and an optic axis 130. At least one of the anterior surface and the posterior surface is aspheric surface and has a toricity configured to spread retroreflected light in a plurality of directions canted from to the optic axis. Stated differently, the anterior surface and/or the posterior surface may be configured to reduce “scary eye”. In addition, the surfaces may be configured to address other conditions in the patient's eye or for other purposes. Thus, the ophthalmic device 100, 100′, 100″, or 100″′ may be selected in step 202. In some embodiments, another ophthalmic device including one or more of the characteristics of the ophthalmic device(s) 100, 100′, 100″, and/or 100″′ may be used.

The ophthalmic device 100 is implanted in the patient's eye, via step 204. Step 204 may include replacing the patient's own lens with the ophthalmic device 100 or augmenting the patient's lens with the ophthalmic device. Treatment of the patient may then be completed. In some embodiments implantation in the patient's other eye of another analogous ophthalmic device may be carried out.

Using the method 200, the ophthalmic device(s) 100, 100′, 100″, 100″′ and/or ophthalmic device may be used. Thus, the benefits of one or more of the transducers 100, 100′, 100″, and/or 100″′may be achieved.

We claim:

1. An ophthalmic device comprising:
an ophthalmic lens having an anterior surface, a posterior surface and an optic axis, at least one of the anterior surface and the posterior surface being an aspheric surface having a first toricity configured to spread reflected light in a plurality of directions not parallel to the optical axis and the other of the posterior or anterior surface having a second toricity at least partially opposite the first toricity.

2. The ophthalmic device of claim 1 wherein the aspheric surface further has a first meridian having a first radius of curvature along a first meridian and a second radius of curvature along a second meridian.

3. The ophthalmic device of claim 2 wherein the first meridian is substantially perpendicular to the second meridian.

4. The ophthalmic device of claim 1 wherein an other of the anterior surface and the posterior surface has an additional toricity opposite to the first toricity such that the ophthalmic lens refracts light as a substantially spherical lens.

5. The ophthalmic device of claim 1 wherein an other of the anterior surface and the posterior surface has an additional toricity such that the ophthalmic lens refracts light as a toric lens.

6. The ophthalmic device of claim 5 wherein the toricity in combination with the additional toricity is configured to correct for an astigmatism of a patient.

7. The ophthalmic device of claim 1 wherein the aspheric surface has a total toricity corresponding to the toricity and an additional toricity, the total toricity configured to correct for an astigmatism of a patient.

8. The ophthalmic device of claim 1 further comprising:
a plurality of haptics coupled with the ophthalmic lens.

9. An ophthalmic device comprising:
an ophthalmic lens having an anterior surface, a posterior surface and an optic axis, the anterior surface having a toricity configured to spread retroreflected light incident to the ophthalmic lens in a direction substantially parallel to the optic axis, the posterior surface having an additional toricity such that the ophthalmic lens has a total toricity configured to correct an ophthalmic condition of a patient; and
a plurality of haptics coupled with the ophthalmic lens.

10. The ophthalmic device of claim 9 wherein the ophthalmic condition is an astigmatism of the patient.

11. A method for treating an ophthalmic condition in a patient comprising:
selecting an ophthalmic device for implantation in an eye of the patient, the ophthalmic device including an ophthalmic lens having an anterior surface, a posterior surface and an optic axis, at least one of the anterior surface and the posterior surface being an aspheric surface having a toricity configured to spread retroreflected light in a plurality of directions canted from to the optic axis; and
implanting the ophthalmic device in the eye of the patient.

12. The method of claim 11 wherein the aspheric surface further has a first meridian having a first radius of curvature along a first meridian and a second radius of curvature along a second meridian.

13. The method of claim 12 wherein the first meridian is substantially perpendicular to the second meridian.

14. The method of claim 11 wherein an other of the anterior surface and the posterior surface has an additional toricity opposite to the first toricity such that the ophthalmic lens refracts light as a substantially spherical lens.

15. The method of claim 11 wherein an other of the anterior surface and the posterior surface has an additional toricity such that the ophthalmic lens refracts light as a toric lens.

16. The method of claim 15 wherein the toricity in combination with the additional toricity are configured to correct for an astigmatism of a patient.

17. The method of claim 11 wherein the aspheric surface has a total toricity corresponding to the toricity and an additional toricity, the total toricity configured to correct for an astigmatism of a patient.

18. The method device of claim 11 wherein the ophthalmic device further includes:
a plurality of haptics coupled with the ophthalmic lens.
